(19) United States
(12) Patent Application Publication
(10) Pub. No.: US 2013/0041430 A1
(43) Pub. Date: Feb. 14, 2013

(54) SYSTEM AND METHOD FOR TREATING A PELVIC CONDITION OF A PATIENT

(71) Applicants: Guangjian Wang, Falcon Heights, MN (US); Brian P. Watschke, Eden Prairie, MN (US); William J. Rissmann, Deephaven, MN (US)

(72) Inventors: Guangjian Wang, Falcon Heights, MN (US); Brian P. Watschke, Eden Prairie, MN (US); William J. Rissmann, Deephaven, MN (US)

(73) Assignee: AMS RESEARCH CORPORATION, Minnetonka, MN (US)

(21) Appl. No.: 13/577,962

(22) PCT Filed: Feb. 4, 2011
(86) PCT No.: PCT/US11/23677

§ 371 Date: Oct. 23, 2012

Related U.S. Application Data
(60) Provisional application No. 61/303,155, filed on Feb. 10, 2010.

(51) Int. Cl. A61N 1/36

(52) U.S. Cl. 607/62

(57) ABSTRACT

A system for treating a pelvic condition of a patient includes a plurality of electrodes, a memory a plurality of predefined unique stimulation programs stored in the memory, and an implantable controller. Each of the stimulation programs defines a stimulation signal and an identification of one or more of the electrodes to which the stimulation signal is to be applied. The implantable controller is electrically coupled to the plurality of electrodes and is configured to execute a selected stimulation program from the memory to apply a stimulation signal to one or more of the electrodes in accordance with the selected stimulation program.

### BACKGROUND

Embodiments of the invention generally relate to systems and methods for treating a pelvic condition of a patient and, more specifically for executing stimulation operations on pelvic tissue of the patient.

Electrical stimulation systems have been used to deliver electrical stimulation therapy to patients to treat a variety of pelvic symptoms or pelvic conditions such as urinary incontinence. A typical electrical stimulation system includes one or more implantable medical leads coupled to an external or implantable electrical stimulator. The implantable medical lead may be implanted in a patient such that at least one stimulation electrode is positioned at a target stimulation site. The one or more electrodes deliver electrical stimulation therapy to the target stimulation site in the form of electrical waveforms or signals.

Current methods for implanting electrodes in pelvic neuromuscular structure (i.e., pelvic tissue such as a pelvic floor muscle), generally involve implanting one or more electrodes in the pelvic neuromuscular structure of the patient and connecting the leads of the electrodes to an external stimulator. Electrical waveforms are then applied to the pelvic neuromuscular structure through the electrodes using the stimulator for the purpose of treating the pelvic condition. The patient wears the external stimulator for a testing period (e.g., two weeks) to observe the effectiveness of the stimulation treatment. If the stimulation treatment appears to be ineffective, an adjustment to the treatment can be made. Once the stimulation of the neuromuscular structure of the patient satisfactorily treats the pelvic condition of the patient, the stimulator device is implanted in the patient.

### SUMMARY

Embodiments of the invention generally relate to systems and methods for treating a pelvic condition of a patient including the execution of stimulation operations on pelvic tissue of the patient. One embodiment of the system includes a plurality of electrodes, a memory, a plurality of predefined unique stimulation programs stored in the memory, and an implantable controller. Each of the stimulation programs defines a stimulation signal and an identification of one or more of the electrodes to which the stimulation signal is to be applied. The implantable controller is electrically coupled to the plurality of electrodes and is configured to execute a selected stimulation program from the memory to apply a stimulation signal to one or more of the electrodes in accordance with the selected stimulation program.

In one embodiment of the method, a system is provided that comprises a plurality of electrodes, a memory, a plurality of predefined unique stimulation programs stored in the memory and at least one processor. Each of the stimulation programs defines a stimulation signal and an identification of one or more of the electrodes to which the stimulation signal is to be applied. In the method, a first stimulation program is selected from the memory using the at least one processor. The first stimulation program is then executed using the at least one processor to apply a stimulation signal to one or more of the electrodes in accordance with the first program.

Other features and benefits that characterize embodiments of the present invention will be apparent upon reading the following detailed description and review of the associated drawings.

### BRIEF DESCRIPTION OF THE DRAWINGS

FIG. 1 is a schematic diagram of a system in accordance with embodiments of the invention.

FIGS. 2 and 3 are side plan views of exemplary electrodes in accordance with embodiments of the invention.

FIGS. 4 and 5 are flowcharts illustrating methods in accordance with embodiments of the invention.

### DETAILED DESCRIPTION OF ILLUSTRATIVE EMBODIMENTS

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure or described herein.

Embodiments of the invention generally relate to systems and methods for treating a pelvic condition of a patient including the execution of stimulation operations on pelvic tissue of the patient. In one embodiment, the systems and methods operate to evaluate the stimulation operations and determine an effective stimulation operation for treating the pelvic condition of the patient. Embodiments of the pelvic condition include, for example, urinary incontinence, fecal incontinence, urinary retention, pelvic pain, sexual dysfunction, endometriosis, interstitial cystitis, chronic prostatitis and other pelvic conditions or symptoms.

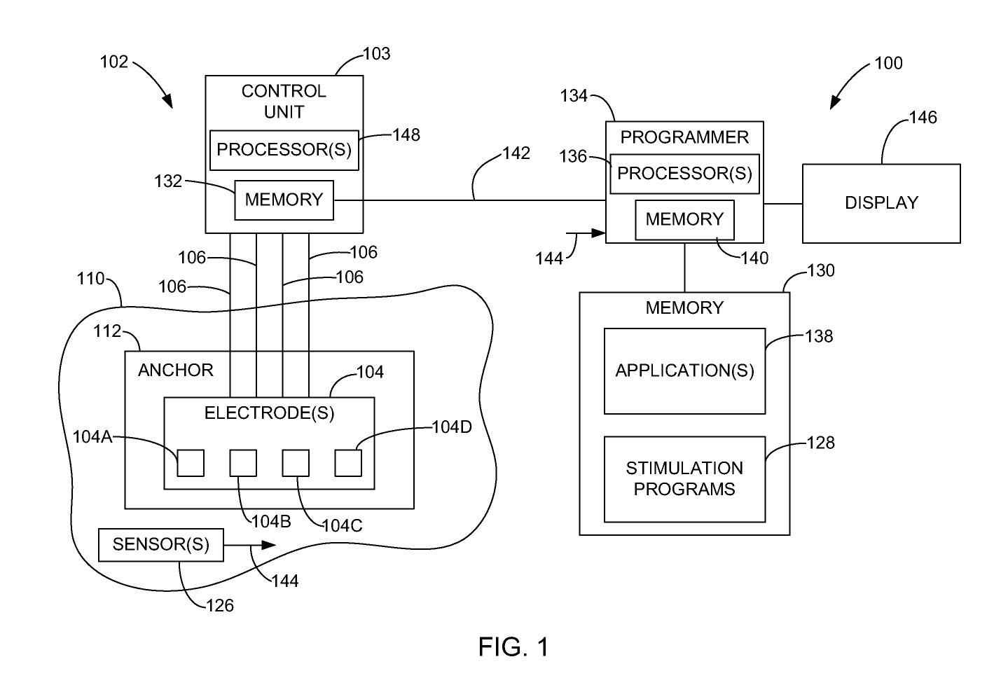

FIG. 1 is a schematic diagram of a system 100 in accordance with embodiments of the invention. One embodiment of the system 100 includes a stimulation device 102. In one embodiment, the stimulation device 102 is configured for surgical implantation in a patient, such as in the pelvic or abdominal region of a patient. That is, an incision is made in the patient, through which the device 102 is inserted. Thus, “surgically implanting” a device in a patient does not describe the placement of the device within a cavity of the patient (e.g., vagina) without making an incision in the patient.

In one embodiment, the device 102 comprises a control unit 103 and one or more implantable electrodes 104, such as electrodes 104A-D. The electrodes 104 are coupled to the control unit or controller 103 by one or more leads 106. Alternatively, the electrodes 104 may be coupled to the control unit 103 through a wireless communication link. The control unit 103 includes one or more processors for executing program instructions, communicating data, analyzing signals, and/or performing other processes as described herein.

The electrodes 104 are configured for implantation in pelvic neuromuscular structure (i.e., pelvic tissue) 110 of the patient, such as a pelvic floor muscle of the patient including the internal urinary sphincter, the external urinary sphincter, the anal sphincter, levator ani and other pelvic tissue 110. In one embodiment, an anchor 112 is attached to the electrodes 104 and anchors the electrodes 104 to the pelvic neuromuscular structure 110.

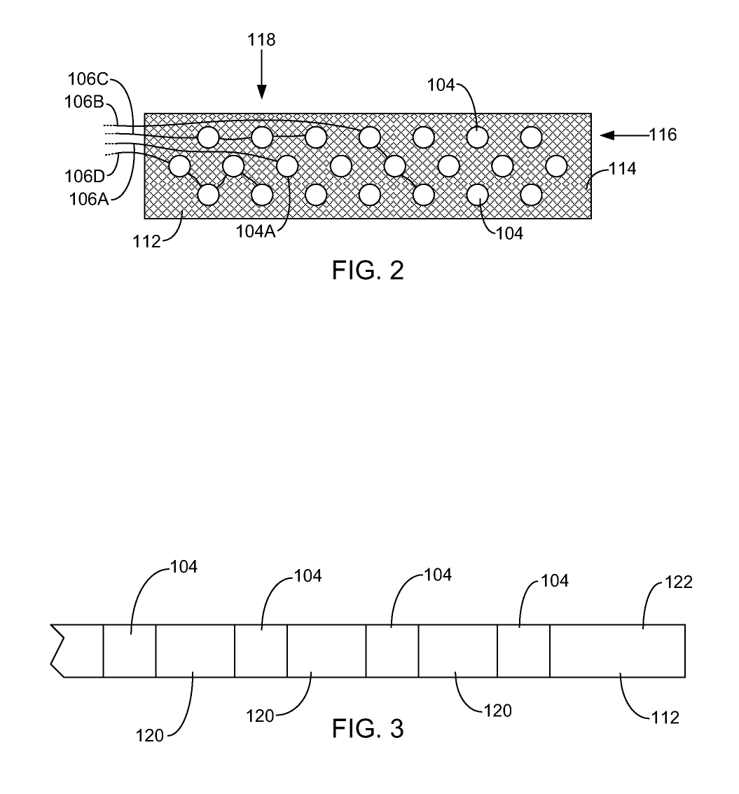

FIGS. 2 and 3 are simplified plan views of exemplary electrodes 104 and anchors 112, in accordance with embodiments in the invention. In one embodiment, the anchor 112 comprises a biocompatible mesh 114, as shown in FIG. 2. In one embodiment, the mesh 114 has a porosity that encourages tissue in-growth through the mesh 114 to anchor the mesh 114 to the tissue 110. In one embodiment, the plurality of the electrodes 104 are attached to the mesh 114. In one embodiment, the electrodes 104 are dispersed over the mesh 114. In one embodiment, the plurality of electrodes are oriented in at least one row extending generally in a lengthwise direction (indicated by arrow 116) across the mesh 114. In one embodiment, the electrodes 104 are oriented in at least one column extending generally in a widthwise direction (indicated by arrow 118) across the mesh 114. In accordance with another embodiment, the electrodes 104 are organized on the mesh 114 in the form of an array comprising a plurality of the rows and columns of the electrodes 104, as shown in FIG. 2. The columns and rows of the electrodes 104 may be oriented perpendicularly to each other, or generally transverse to each other as shown in FIG. 2.

In one embodiment, the conductive leads 106 are attached to the electrodes 104 on the mesh 114 and electrically couple the electrodes 104 to the control unit 103. In one embodiment, each lead 106 is attached to one or more of the electrodes 104. This allows for the selective application of stimulation signals from the control unit 103 to a single electrode 104, a group of electrodes 104, and/or other combinations of the electrodes, through a single lead 106. For instance, exemplary lead 106A is electrically coupled to electrode 104A. Thus, a stimulation signal conducted through the lead 106A delivers the signal to the single electrode 104A. Exemplary leads 106B-D electrically couple the control unit 103 to a plurality of the electrodes 104 to allow the control unit 103 to apply a stimulation signal to multiple electrodes 104 simultaneously through a single lead 106. Lead 106B electrically couples an exemplary column of electrodes 104 to the control unit 103, lead 106C electrically couples an exemplary row of electrodes 104 to the control unit 103, and lead 106D electrically couples an exemplary group or cluster of electrodes 104 to the control unit 103. Additional leads 106 coupled to the remaining electrodes 104 are not shown in FIG. 2 in order to simplify the illustration.

In the embodiment illustrated in FIG. 3, the electrodes 104 are arranged in a row and are separated from each other by electrically insulative material 120. Leads 106 (not shown) are each electrically coupled to one or more of the electrodes 104 to deliver stimulation signals from the control unit 103 to a single electrode 104 or a group of the electrodes 104. An anchor 112, such as a helical coil or other conventional anchor, is attached at a distal end 122 to anchor the electrodes 104 to the pelvic tissue 110, in which they are implanted.

As discussed above, embodiments of the device 102 allow for stimulation operations to be performed on the tissue 110 in which stimulation signals (e.g., electrical waveforms) are selectively applied to one of the electrodes 104, a subset of the plurality of the electrodes 104, or all of the electrodes 104. The application of the stimulation signal to two or more electrodes 104 may be through multiple leads 106, each of which electrically couples one or more electrodes 104 to the control unit 103, or through a single lead 106 that electrically couples the two or more electrodes 104 to the control unit 103. Thus, the stimulation device 102 is configured to apply an electrical waveform or stimulation signal to a subset of the electrodes 104, such as the single electrode 104B or a group of electrodes 104, such as 104B and 104D, shown in FIG. 1. Due to the separation of the electrodes 104, the application of an electrical signal to one of the electrodes 104 may have a different effect on the pelvic neuromuscular structure 110 than an electrical waveform applied through an adjacent electrode 104. As a result, by selectively activating the individual electrodes 104, or groups of the electrodes 104, one can stimulate different portions of the pelvic neuromuscular structure 110 and determine which of the electrodes 104 is best suited for delivering the stimulation signal for treatment of the pelvic condition of the patient.

In one embodiment, the device 102 includes at least one physiological sensor 126, shown in FIG. 1, which is configured to sense a physiological parameter of the patient and assist in the evaluation of the effectiveness of a stimulation operation on the tissue 110 by the device 102. One embodiment of the sensor 126 includes one or more of the electrodes 104, which can conduct, for example, electromyographic (EMG) signals from the tissue 110, to the control unit 103 through the leads 106. In accordance with other embodiments, the physiological sensor 126 comprises a pressure sensor, a motion sensor and/or an acceleration sensor. In one embodiment, the sensor 126 is implanted in the pelvic region of the patient. In one embodiment, the sensor 126 is in contact with the pelvic tissue 110.

In one embodiment, the control unit 103 is configured to execute a stimulation program to perform a stimulation operation on the tissue 110 of the patient. The stimulation program defines the stimulation operation including a stimulation signal and an identification of one or more of the electrodes 104 to which the stimulation signal is to be applied. In one embodiment, the stimulation programs 128 are stored in memory 130 located external to the control unit 103, as shown in FIG. 1, or memory 132 of the control unit 103. In one embodiment, each of the stimulation programs 128 are unique in that each defines a unique stimulation operation. That is, each of the stimulation programs 128 defines a unique stimulation signal and/or identifies a unique subset of the electrodes 104 relative to the other stimulation programs 128.

Execution of one of the stimulation programs 128 by the control unit 103 performs a stimulation operation in which the stimulation signal defined by the stimulation program is generated and delivered to the one or more identified electrodes through the corresponding leads 106. The stimulation signal is delivered to the tissue 110 through the identified electrodes 104, while the remaining electrodes 104 do not deliver the stimulation signal (i.e., are not activated), but remain available to operate as physiological sensors 126 that receive the electromyographic signal generated in response to the applied stimulation signal.

The stimulation signal is designed to treat a pelvic condition of the patient and is defined by stimulation parameters. Embodiments of the stimulation parameters include, for example, a current magnitude, a voltage magnitude, a duty cycle, a pulse width, a polarity, and/or a pulse repetition rate or frequency of the electrical pulses. Exemplary embodiments of the stimulation signal include a current amplitude of 2-100 mA. In one embodiment, one or more of the stimulation signals defined by the stimulation programs have a current amplitude within a range of approximately 2-4 mA. Exemplary embodiments of the voltage amplitude of the stimulation signals defined by the stimulation programs include 1-15 volts with a preferred range of 1-6 volts. Exemplary embodiments of the pulse width of the stimulation signals defined by the stimulation programs include 10 μs-2 ms with a preferred range of 100-200 μs. Exemplary embodiments of the pulse repetition rate or frequency for the stimulation signals defined by the stimulation programs include ranges of 5-50 Hz, 12-25 Hz, 50-1000 Hz, with a preferred range of 10-30 Hz.

Additional embodiments of the stimulation parameters include a variable current amplitude, which determines a rate that the current amplitude is either increased or decreased from a starting amplitude. Similarly, another embodiment of the stimulation parameters includes a variable voltage amplitude, which determines a rate that the voltage either increases or decreases from a starting voltage amplitude. Another embodiment of the stimulation parameters includes a set period of time to delay application of the electrical waveform from the last time the electrical waveform was applied through the one or more electrodes 104. The stimulation parameters can also include square and sine wave signal patterns for the electrical waveform. Other stimulation parameters can also be defined in the stimulation programs.

In one embodiment, the system 100 includes a programmer 134, which can be used to program the control unit 103 to executed one or more of the stimulation programs 128, and/or evaluate the effectiveness of stimulation operations in treating the pelvic condition of the patient. In one embodiment, the programmer 134 comprises one or more processors 136 that execute instructions of one or more applications, such as application 138 stored, for example, in the memory 130, to perform one or more of the tasks described below. In one embodiment, an operator of the system 100 may configure the programs 128 using the programmer 134. The programmer 134 can be in the form of a handheld device, or a device that is controlled through a computer running a host application, for example.

In one embodiment, the programmer 134 includes a memory 140, in which the applications 138 and/or the stimulation programs 128 may be stored for execution by the one or more processors 136. In another embodiment, the programmer 134 is configured to access the memory 130 using conventional data access techniques. In one embodiment, the programmer 134 delivers one or more of the stimulation programs 128 to the control unit 103 through a communication link 142, which may be a physical communication link (e.g., a cable) or a wireless communication link (e.g., radio frequency). In one embodiment, the programmer 134 is configured to receive information from the control unit 103 through the communication link 142, such as information contained in the memory 132. In one embodiment, the programmer 134 is configured to receive information from the one or more sensors 126, such as sensed physiological parameters of the patient. This sensor information may be in the form of a raw or processed sensor signal 144 generated by the sensor 126, or other data generated based on the signal 144, such as information obtained through the processing of the signal 144 by the control unit 103, for example.

In one embodiment, the programmer 134 is configured to provide information on a display 146. This information may include a graphical user interface for operating the programmer 134, information relating to the one or more sensed physical parameters by the one or more physiological sensors 126, information relating to the device 102, and/or other information.

As mentioned above, one embodiment of the programmer 134 is configured to execute instructions of the one or more applications 138 (hereinafter “application”) stored in the memory 130 and/or the memory 140 to perform method steps described herein. In accordance with another embodiment, the application 138 is stored in the memory 132 of the control unit 103. In one embodiment, the control unit 103 comprises one or more processors 148 that are configured to execute the application stored in the memory 132 to perform method steps described herein. Thus, while the exemplary embodiments of the methods are described below as being performed specifically by the programmer 134, it is understood that embodiments of the methods include the performance of steps by the control unit 103 responsive to the execution of the application stored in the memory 132. The execution of the application by either the programmer 134 or the control unit 103 is performed by one or more processors of those devices.

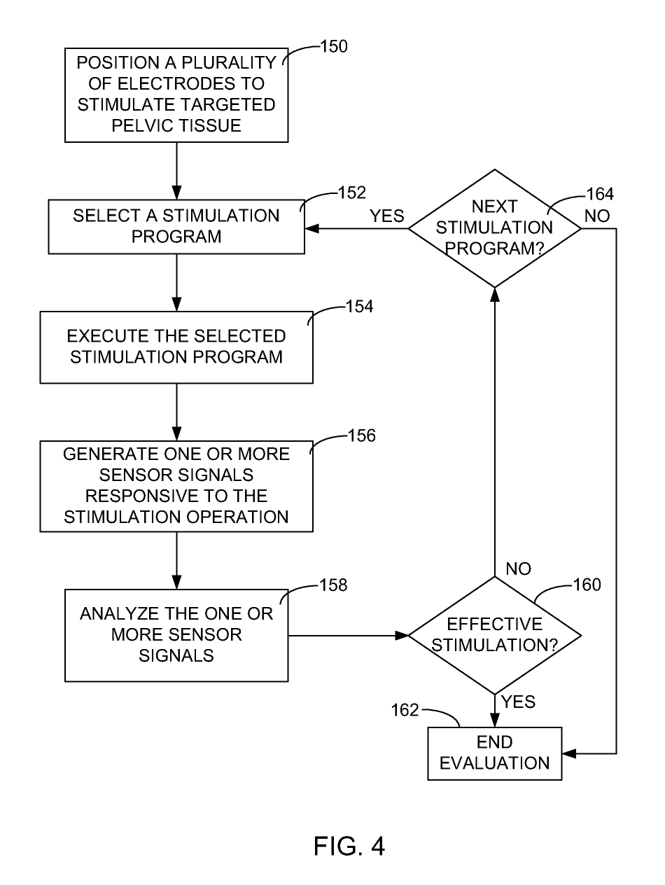

In one embodiment, the execution of the application 138 performs embodiments of the method illustrated in the flowchart of FIG. 4. The execution of the application 138 may include input from an operator of the system 100 to perform the methods described herein. For instance, the operator may configure the various stimulation programs, select the initial stimulation program, define the adjustments to be made to the initial stimulation program, and/or provide other input to the application 138 through, for example, an interface provided by the programmer 134.

In one embodiment, the system 100 in accordance with one or more embodiments described herein is provided. In one embodiment, of the method, the one or more electrodes 104 are positioned to stimulate targeted pelvic tissue 110 of the patient, at 150. In one embodiment of step 150, the one or more electrodes 104 are surgically implanted in the patient in accordance with conventional techniques. In one embodiment, the electrodes 104 are surgically implanted in the patient such that at least one of the electrodes is in physical contact (e.g., implanted within) with the pelvic tissue 110.

Next, at 152, the operator of the programmer 134 or the executed application 138 selects one of the stimulation programs 128. As mentioned above, in one embodiment, each of the stimulation programs 128 are unique in that they define a unique stimulation operation comprising a stimulation signal having unique stimulation parameters and/or the identification of a unique set of the electrodes 104, to which the stimulation signal will be applied. In one alternative embodiment, each of the stimulation programs 128 identify the same set of the electrodes 104 for stimulation, but define a unique stimulation signal will be applied to the set of the electrodes 104. In yet another embodiment, the stimulation programs 128 each define the same stimulation signal, but identify different sets of the one or more electrodes 104.

At 154 of the method, the selected stimulation program 128 is executed by the control unit to perform a stimulation operation on the tissue 110 in accordance with the selected stimulation program 128. As discussed above, the stimulation operation delivers a stimulation signal having stimulation parameters defined by the selected stimulation program to the pelvic tissue 110 through the one or more electrodes 104 identified by the selected stimulation program.

In one embodiment, one or more sensor signals 144 each relating to a physiological parameter of the patient are generated, at 156, by the one or more physiological sensors 126, and/or one or more of the electrodes 104, in response to the stimulation operation of step 154. In one embodiment, the one or more signals 144 are recorded (i.e., stored in memory, such as memory 130, 132 or 140) and can be accessed by the programmer 134 for processing. In one embodiment, the signals 144 undergo processing prior to their delivery to the programmer 134 or their storage in memory to place them in a desired format, eliminate noise and other undesired signals, and/or perform other conditioning of the signals 144. In one embodiment, the received signals 144 are presented on the display 146.

In one embodiment, the programmer 134 (i.e., the executed application 138) analyzes the one or more sensor signals 144, at 158, and determines the effectiveness of the executed stimulation operation on the pelvic tissue 110 and/or the condition of the patient targeted for treatment by the electrical stimulation. Exemplary properties of the one or more signals 144 that are detected or measured in step 158 to assist in the evaluation of the stimulation operation include: a change in amplitude of the electrical activity generated by the tissue 110 following the stimulation operation; a change in frequency content of the electrical activity generated by the tissue 110 responsive to the stimulation operation; a change in the pulse width of the electrical signals generated by the tissue 110 or surrounding pelvic floor muscles before, after and/or during the stimulation operation; the time between the execution of the stimulation operation and an evoked response from the tissue 110; and/or other properties of the signals 144.

In one embodiment, the signals 144 are EMG signals sensed by the sensor 126 or one or more of the electrodes 104. In one embodiment of step 158, these signals are analyzed to determine the patient's intrinsic electrical activity for the applied stimulation signal. In one embodiment, the electrical activity generated by the tissue 110 responsive to the application of stimulation signal is analyzed across the various electrodes 104 to determine the primary electrical vector and to determine if the stimulation pulse resulted in a change in the primary electrical vector.

In one embodiment, if the stimulation operation was determined to be effective at 160, the evaluation is terminated at 162 and the control unit 103 can be programmed to execute the selected stimulation program in the treatment of the pelvic condition of the patient. In one embodiment, the programmed stimulation device 102 is implanted in the patient.

In one embodiment, if it is determined at 160 that the stimulation performed by the control unit 103 in accordance with the selected stimulation program was ineffective in generating the desired physiological response or treating the targeted condition of the patient, the method moves to 164, where it is determined whether additional predefined stimulation programs have yet to be tested. If there are unexecuted stimulation programs 128, the method returns to step 152 where one of the unexecuted stimulation programs 128 is selected. The selected stimulation program 128 is then executed at 154 to deliver a new stimulation operation to the tissue 110. At 158, the method analyzes the effect of the stimulation operation based on the one or more signals 144, as discussed above. In one embodiment, the method ends at 162 if the stimulation operation was determined to be effective at 160, and continues with step 164 to determine whether there are unexecuted stimulation programs to try if the stimulation operation was ineffective. If all of the stimulation programs have been executed, the method ends at 162.

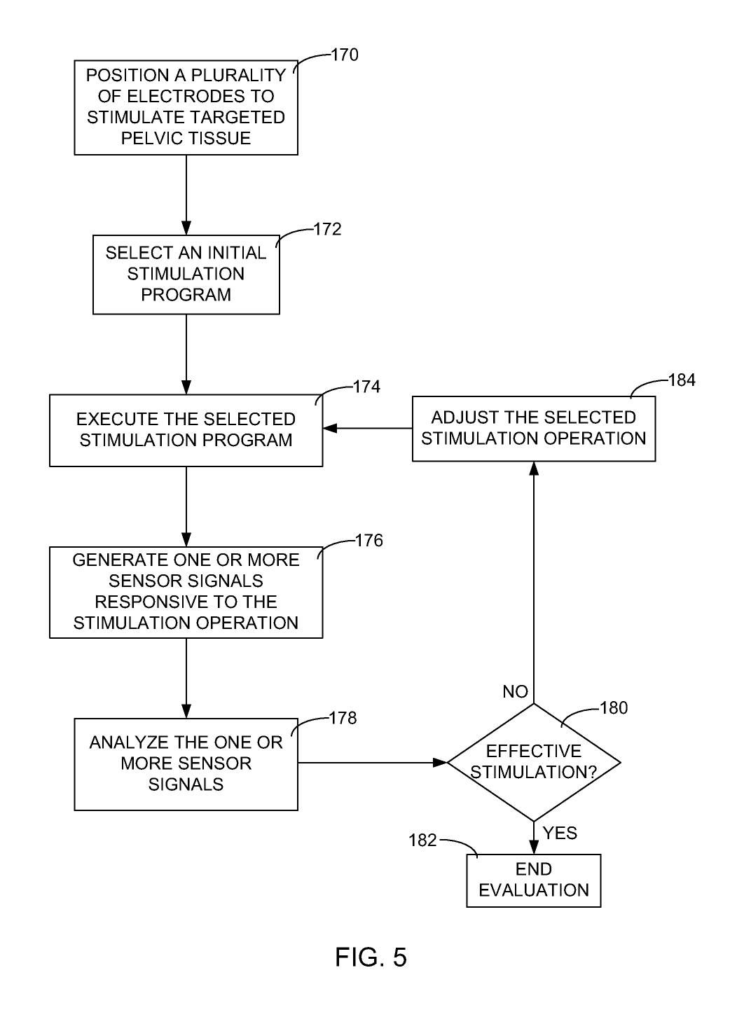

FIG. 5 is a flowchart illustrating a method in accordance with additional embodiments of the invention that is performed responsive to execution of the application 138. In one embodiment, the system 100 in accordance with one or more embodiments described herein is provided. In one embodiment, the one or more electrodes 104 are positioned to stimulate targeted pelvic tissue 110 of the patient, at 170. In one embodiment of step 170, the one or more electrodes 104 are surgically implanted in the patient in accordance with conventional techniques. In one embodiment, the electrodes 104 are surgically implanted in the patient such that at least one of the electrodes is in physical contact (e.g., implanted within) with the pelvic tissue 110.

At 172, rather than selecting among multiple stimulation programs as discussed above, an initial stimulation program is selected from memory, such as memory 130, 132 or 140. The initial stimulation program defines an initial stimulation signal defined by stimulation parameters in accordance with embodiments described above. In one embodiment, the initial stimulation program identifies one or more of the electrodes 104, to which the initial stimulation signal is to be applied upon execution of the initial stimulation program by the control unit 103. In accordance with another embodiment, the one or more electrodes are selected by the operator of the system 100.

At 174, the initial stimulation program is executed by the control unit 103 to perform a stimulation operation on the tissue 110 in accordance with the selected stimulation program, as discussed above with regard to step 154 of the method of FIG. 4. In one embodiment, one or more sensor signals 144 each relating to a physiological parameter of the patient are generated, at 176, by the one or more physiological sensors 126, and/or one or more of the electrodes 104, in response to the stimulation operation of step 174 in accordance with the embodiments described above with regard to step 156. In one embodiment, the signals 144 are presented on the display 146.

At 178, the programmer 134 (i.e., the executed application 138) analyzes the one or more sensor signals 144 and determines the effectiveness of the executed stimulation operation on the pelvic tissue 110 and/or the condition of the patient targeted for treatment by the electrical stimulation, in accordance with the embodiments described above with regard to step 158. In one embodiment, if the stimulation operation is determined to be effective at 180, the method ends at 182 and the control unit 103 can be programmed accordingly by, for example, storing the stimulation program 128 in memory 132.

In one embodiment, if the stimulation operation was not determined to be effective at 180, the method adjusts the stimulation operation of the selected stimulation program 128, at 184, to form a new selected stimulation program. In one embodiment of step 184, one or more of the stimulation parameters of the stimulation signal and/or the identified (i.e., active) electrodes 104 of the selected stimulation program 128 are adjusted. Embodiments of the adjustment to the one or more stimulation parameters include modifications to one or more of the stimulation parameters discussed above by a predefined amount (i.e., delta change), or a change of one or more of the stimulation parameters to a corresponding predefined value. The delta change value may be set by the application 138 or the operator of the system 100. In one embodiment, the predefined value is extracted from a list of values stored in memory (such as memory 130, 132 or 140).

In one embodiment of step 184, the adjustment to the active electrodes involves selecting one or more of the electrodes 104 in accordance with a predefined routine. Such a routine may involve selecting each of the electrodes 104 individually over a series of adjustment steps 184, selecting different clusters (e.g., groups, rows, columns, etc.) of the electrodes 104 over a series of adjustment steps 184, or other predefined pattern of adjusting the active electrodes.

The adjusted stimulation program becomes the new selected stimulation program that is executed at 174 by the control unit 103. This results in the performance of a stimulation operation in accordance with the adjusted stimulation program as discussed above. The method then continues as discussed above.

In one embodiment, the adjustment step 184 is executed a limited number of times. In one embodiment, if none of the stimulation operations executed at 174 are found to be effective, the evaluation ends.

In one embodiment, the signals 144 or other data representing the one or more physiological parameters are stored in memory, such as memory 130 after each stimulation program or stimulation operation is executed (step 154, step 174). In one embodiment, the analysis of the one or more physiological parameters that occurs at steps 158 and 178 is delayed until after the execution of a predefined number of the selected stimulation programs. In one embodiment, the analysis determines the effectiveness of the executed stimulation programs or stimulation operations based on the one or more physiological parameters as discussed above, and determines the most effective stimulation program for treating the condition of the patient. The most effective stimulation program is then programmed into the controller 103. In one embodiment, this selection of the most effective stimulation program is performed by the application 138. In another embodiment, a clinician selects the most effective stimulation program or operation based on the results of the analysis steps 158 and 178, which may be presented on the display 146.

After the control unit 103 is programmed to execute a desired stimulation program and is implanted in the patient, it may be necessary to adjust the stimulation program due to changes in the placement of the electrodes 104, changes in the tissue 110, or other changes. In one embodiment, the controller 103 periodically executes embodiments of the methods described above through the execution of the application 138 stored in the memory 132 to determine an effective stimulation operation or program for treating the condition of the patient. As a result, modifications can be periodically made to the stimulation program automatically by the control unit 103 thereby adjusting the stimulation therapy as the patient goes about his or her daily activities. Alternatively, the control unit 103 can communicate the results of the evaluation method (e.g., recorded physiological parameters or signals 144) for review by a clinician through, for example, the programmer 134. The control unit 103 can then be programmed as desired by the clinician based on the test results.

In one embodiment, the programmer 134 is directly coupled to the electrodes and executes the stimulation program or operation to deliver the selected stimulation signal to the tissue 110 through the active electrodes 104 during the evaluation methods described above. Thus, in one embodiment, the control unit 103 is not used to perform the execution steps 154 or 174, the generation steps 156 or 176, or the analyzing steps 158 and 178. In one embodiment, the one or more electrodes 104 are used as the physiological sensors for generating the signals 144. After the effective stimulation program is determined, the programmer 134 programs the control unit 103 accordingly. The electrodes 104 are then electrically coupled to the control unit 103 and the control unit 103 is implanted in the patient, possibly after undergoing testing.

Although the present invention has been described with reference to preferred embodiments, workers skilled in the art will recognize that changes may be made in form and detail without departing from the spirit and scope of the invention.

1. A system for treating a pelvic condition of a patient comprising:
a plurality of electrodes;
a memory;
a plurality of predefined unique stimulation programs stored in the memory, each stimulation program defining a stimulation signal and an identification of one or more of the electrodes to which the stimulation signal is to be applied;
an implantable controller electrically coupled to the plurality of electrodes and configured to execute a selected stimulation program from the memory to apply a stimulation signal to one or more of the electrodes in accordance with the selected stimulation program; and
a programmer comprising a processor configured to identify differences in sensed physiological parameters of the patient detected responsive to the execution of two or more of the stimulation programs by the controller.

2. The system of claim 1, wherein the stimulation programs define at least two unique stimulation signals, each having a unique set of stimulation parameters.

3. The system of claim 2, wherein the stimulation parameters are selected from the group consisting of a current amplitude, a voltage amplitude, a polarity, a duty cycle, a pulse width, a pulse frequency, a starting current amplitude and a rate of change of the current amplitude, a starting voltage amplitude and a rate of change of the voltage amplitude, a set period of time to delay application of the stimulation signal from the last time the stimulation signal was applied, and a signal pattern.

4. The system of claim 1, wherein the stimulation programs identify at least two unique subsets of the plurality of electrodes to which the stimulation signal is to be applied.

5. The system of claim 1, further comprising a physiological sensor electrically coupled to the controller, the physiological sensor configured to sense-a the physiological parameter of the patient.

6. The system of claim 5, wherein the physiological sensor is selected from the group consisting of at least one of the electrodes, a pressure sensor, a motion sensor and an acceleration sensor.

7. The system of claim 5, wherein the physiological parameter is selected from the group consisting of an electromyographic signal, a pressure, a motion and an acceleration.

8. (canceled)

9. The system of claim 1, wherein the controller comprises the memory.

10. The system of claim 1, wherein the programmer is configured to access the memory and communicate a selected stimulation program from the memory to the controller through one of a physical communication link and a wireless communication link.

11. A method of treating a pelvic condition of a patient comprising:
providing a system comprising:
a plurality of electrodes;
a memory;
a physiological sensor;
a plurality of predefined unique stimulation programs stored in the memory, each stimulation program defining a stimulation signal and
a unique set of the plurality of electrodes to which the stimulation signal is to be applied; and
at least one processor;
separately executing each of the plurality of stimulation programs using the at least one processor comprising applying the a stimulation signals to the sets of electrodes in accordance with the each of the program;
analyzing a physiological response to the execution of each of the stimulation programs using the physiological sensor and the each one processor;
selecting from the executed stimulation programs a final stimulation program based on analyzing a physiological response; and
programming an implantable control unit to execute the final stimulation program.

12-13. (canceled)

14. The method in accordance with claim 10, wherein each of the stimulation programs defines a stimulation signal having unique stimulation parameters.

15. The method in accordance with claim 11, wherein the stimulation parameters are selected from the group consisting of a current amplitude, a voltage amplitude, a polarity, a duty cycle, a pulse width, a pulse frequency, a starting current amplitude and a rate of change of the current amplitude, a starting voltage amplitude and a rate of change of the voltage amplitude, a set period of time to delay application of the stimulation signal from the last time the stimulation signal was applied, and a signal pattern.

16. The method in accordance with claim 10, wherein analyzing a physiological response to the execution of each of the stimulation programs comprises receiving a physiological response from the physiological sensor responsive to the execution of each of the stimulation programs.

17-18. (canceled)

19. The method in accordance with claim 10, further comprising:
surgically implanting the control unit in the patient;
surgically implanting the one or more electrodes in contact with pelvic tissue of the patient;
executing the final stimulation program using the controller comprising applying a stimulation signal to the set of electrodes in accordance with the final stimulation program; and
treating the pelvic condition of the patient responsive to executing the final stimulation program.

20. The method in accordance with claim 44, wherein the condition of the patient is selected from the group consisting of urinary incontinence, fecal incontinence, urinary retention, pelvic pain, sexual dysfunction, endometriosis, interstitial cystitis, and chronic prostatitis.
